(19) United States
(12) Patent Application Publication
(10) Pub. No.: US 2026/0057976 A1
(43) Pub. Date: Feb. 26, 2026

(54) IMPROVING EXPLAINABILITY OF PATIENT REPRESENTATIONS IN HEALTHCARE AND HOSPITAL MANAGEMENT SYSTEMS

(71) Applicant: NEC Laboratories Europe GmbH, Heidelberg (DE)

(72) Inventors: Francesco ALESIANI, Heidelberg (DE); Giampaolo PILEGGI, Heidelberg (DE); Makoto TAKAMOTO, Heidelberg (DE)

(21) Appl. No.: 19/104,242

(22) PCT Filed: Jul. 10, 2023
(86) PCT No.: PCT/IB2023/057061

§ 371 Date: Feb. 17, 2025

(30) Foreign Application Priority Data
Mar. 17, 2023 (EP) 23162713

(51) Int. Cl. G16H 10/60; G16H 50/30

(52) U.S. Cl. CPC G16H 10/60; G16H 50/30

(57) ABSTRACT

A method for improving explainability of patient representations includes generating one or more patient representations of a patient based on building one or more invariant feature representations of the patient. The one or more patient representations indicate one or more discrete features. The method further includes determining predictions for one or more downstream tasks based on using the one or more discrete features and providing explanations associated with the one or more discrete features. The explanations are associated with the predictions for the one or more downstream tasks.

### CROSS-REFERENCE TO PRIOR APPLICATION

This application is a U.S. National Phase application under 35 U.S.C. § 371 of International Application No. PCT/IB2023/057061, filed on Jul. 10, 2023, and claims benefit to European Patent Application No. EP23162713.4, filed on Mar. 17, 2023, the entire contents of which is hereby incorporated by reference herein. The International Application was published in English on Sep. 26, 2024 as WO 2024/194681 A1 under PCT Article 21 (2).

### FIELD

The present invention relates to artificial intelligence (AI) and machine learning (ML), and in particular to a method, system and computer-readable medium for improving explainability of patient representations including aggregating patient information from various sources and using the aggregated patient information for different prediction systems.

### BACKGROUND

Graph neural networks are modem tools to process multimodal data and to integrate information from various sources. When systems are unable to understand in advance which tasks need to be implemented with the collected data, a mechanism can be used to generate a representation that is generic. In this context, representation learning over graph neural network is a powerful tool. Unfortunately, the generalizability of the representation hinders explainability of the downstream tasks.

Current explainable models requires the access to the full AI model, while in previous presented context, the feature extraction and the prediction tasks are separated, making explainability impossible.

### SUMMARY

In an embodiment, the present disclosure provides a computer-implemented method for improving explainability of patient representations. For instance, one or more patient representations of a patient are generated based on building one or more invariant feature representations of the patient. The one or more patient representations indicate one or more discrete features. Predictions for one or more downstream tasks are determined based on using the one or more discrete features. The explanations associated with the one or more discrete features are provided for display. The explanations are associated with the predictions for the one or more downstream tasks.

### BRIEF DESCRIPTION OF THE DRAWINGS

Embodiments of the present invention will be described in even greater detail below based on the exemplary figures. The present invention is not limited to the exemplary embodiments. All features described and/or illustrated herein can be used alone or combined in different combinations in embodiments of the present invention. The features and advantages of various embodiments of the present invention will become apparent by reading the following detailed description with reference to the attached drawings which illustrate the following:

FIG. 1 illustrates a simplified block diagram depicting an exemplary computing environment according to an embodiment of the present disclosure;

FIG. 2 shows a multi-source, multi-prediction patient representation learning according to an embodiment of the present disclosure:

FIG. 3 shows a high level description of invariant and interpretable graph fingerprints according to an embodiment of the present disclosure:

FIG. 4 shows discrete latent graph feature learning with Invariant (and Interpretable) Graph Fingerprint (I2GF) according to an embodiment of the present disclosure:

FIG. 5 shows dedicated losses that are used to reconstruct the input features or to promote prototypes, counterfactuals, or the basic contrastive loss according to an embodiment of the present disclosure:

FIG. 6 shows invariant representation learning with constative loss and graph perturbation and masking according to an embodiment of the present disclosure:

FIG. 7 shows graph generation policies according to an embodiment of the present disclosure:

FIG. 8 shows visualization of virtual nodes according to an embodiment of the present disclosure:

FIG. 9 shows an alternative architecture for a discrete graph variational auto encoder according to an embodiment of the present disclosure:

FIG. 10 shows a discrete diffusion model according to an embodiment of the present disclosure:

FIG. 11 shows an IGF that is used for prediction of length of stay and to provide additional information (e.g., justification or explanations) to a hospital personnel according to an embodiment of the present disclosure:

FIG. 12 shows an IGF that is used for predicting a disease of the patient in a multiclass classification task using a microbiome database according to an embodiment of the present disclosure; and

FIG. 13 is a block diagram of an exemplary processing system, which can be configured to perform any and all operations disclosed herein.

### DETAILED DESCRIPTION

Effective healthcare evaluates risks of complications by analyzing patient health records. To perform this, clinical personnel and national regulators can deem it necessary for artificial intelligence (AI) to provide explainable predictions and explainable methods. Embodiments of the present invention utilize a new method and system to improve explainability of patient representations.

For instance, embodiments of the present invention describe a method that allows to separate the two steps (e.g., feature extraction and prediction tasks), and still provide explainable information as well as show its application in the healthcare domain, where the patient information is aggregated from various sources and is used for different prediction systems. Therefore, embodiments of the present invention allow for multiple downstream tasks to be performed on the graph representations, without having to execute (e.g., run) the representation learning model that might not have access while still providing explanations of the prediction.

According to a first aspect, the present invention provides a computer-implemented method for improving explainability of patient representations. The method includes generating one or more patient representations of a patient based on building one or more invariant feature representations of the patient. The one or more patient representations indicate one or more discrete features. The method further includes determining predictions for one or more downstream tasks based on using the one or more discrete features. The method also includes providing (e.g., for display) explanations associated with the one or more discrete features. The explanations are associated with the predictions for the one or more downstream tasks.

According to a second aspect, the method according to the first aspect further comprises collecting data from a plurality of patients from different subsystems within a hospital environment; and creating an electronic health record (EHR) database based on the collected data. Further generating the one or more patient representations is based on using the EHR database.

According to a third aspect, the method according to any of the first or the second aspect further comprises that generating the one or more patient representations of the patient comprises generating biomarkers for the patient and determining the predictions for the one or more downstream tasks is based on the generated biomarkers.

According to a fourth aspect, the method according to any of the first to third aspects further comprises training a model based on the biomarkers for the patient. Further, determining the predictions is based on the trained model.

According to a fifth aspect, the method according to any of the first to fourth aspects further comprises: predicting one or more risks for the patient based on using the trained model and detecting, based on the one or more risks, specific biomarkers from the generated biomarkers that cause each of the predictions. The explanations indicate the predictions and the specific biomarkers that caused the predictions.

According to a sixth aspect, the method according to any of the first to fifth aspects further comprises that providing, for display, the explanations comprises providing the explanations for display on a hospital display device associated with hospital personnel, one or more patients, or other users.

According to a seventh aspect, the method according to any of the first to sixth aspects further comprises that the one or more discrete features comprise invariant graph fingerprint (IGF) features, the explanations are associated with the IGF features, and the explanations indicate importance of the IGF features according to Shapley importance explanations.

According to an eighth aspect, the method according to any of the first through seventh aspects further comprises that generating the one or more patient representations of the patient comprises determining a first invariant graph fingerprint (IGF) feature for input features based on using a graph artificial intelligence (Graph AI) and input data. The first IGF feature is a discrete version of the input data.

According to an ninth aspect, the method according to any of the first through eighth aspects further comprises that generating the one or more patient representations of the patient comprises determining, based on using the Graph AI and the input data, a second IGF feature for prediction tasks and a third IGF feature for prototypes. The second IGF feature is a discrete subset of the input data that is used for a prediction of a specific task and the third IGF feature indicates a clustering of the input data associated with similarities between the one or more patient representations.

According to a tenth aspect, the method according to any of the first through ninth aspects further comprises that determining the third IGF feature for prototypes is based on using one or more generated virtual nodes and adding features that are determined using a k-Nearest neighbor algorithm.

According to an eleventh aspect, the method according to any of the first through tenth aspects further comprises that generating the one or more patient representations of the patient comprises determining a fourth IGF feature for counterfactuals and determining a fifth IGF feature for a contrastive associated with a contrastive loss.

According to a twelfth aspect, the method according to any of the first through eleventh aspects further comprises that the contrastive loss is associated with minimizing the Kullback-Leibler (KL) divergence, performing mutual information maximization, and/or maximizing the cosine similarity function.

According to a thirteenth aspect, the method according to any of the first through twelfth aspects further comprising that generating the one or more patient representations of the patient comprises determining one or more IGF features based on using a dedicated loss or one or more unsupervised computations.

According to a fourteenth aspect of the present disclosure, a computer system is provided for improving explainability of patient representations, the system comprising one or more hardware processors, which, alone or in combination, are configured to provide for execution of the following steps: generating one or more patient representations of a patient based on building one or more invariant feature representations of the patient, wherein the one or more patient representations indicate one or more discrete features: determining predictions for one or more downstream tasks based on using the one or more discrete features; and providing (e.g., for display) explanations associated with the one or more discrete features, wherein the explanations are associated with the predictions for the one or more downstream tasks.

A fifteenth aspect of the present disclosure provides a tangible, non-transitory computer-readable medium having instructions thereon, which, upon being executed by one or more processors, provides for execution of the method according to any of the first to the thirteenth aspects and/or the method comprising the following: generating one or more patient representations of a patient based on building one or more invariant feature representations of the patient, wherein the one or more patient representations indicate one or more discrete features: determining predictions for one or more downstream tasks based on using the one or more discrete features; and providing (e.g., for display) explanations associated with the one or more discrete features, wherein the explanations are associated with the predictions for the one or more downstream tasks.

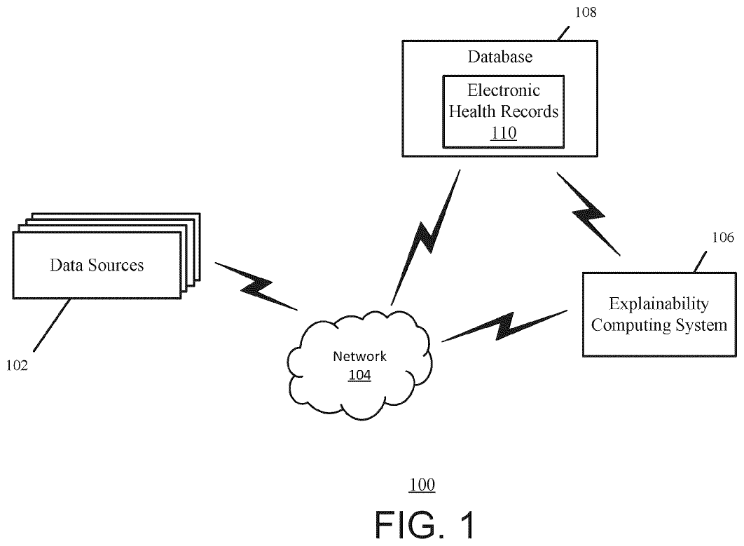

FIG. 1 illustrates a simplified block diagram depicting an exemplary computing environment according to an embodiment of the present disclosure. For instance, FIG. 1 shows a computing environment 100 comprising a plurality of data sources 102, a network 104, an explainability computing system 106, and a database 108. The database 108 stores information such as electronic health records (EHR) 110. Although certain entities within environment 100 are described below and/or depicted in the FIGs. as being singular entities, it will be appreciated that the entities and functionalities discussed herein can be implemented by and/or include one or more entities. For example, in some instances, the explainability computing system 106 can be and/or include multiple computing devices such as a first computing device and a second computing device.

The entities within the environment 100 are in communication with other devices and/or systems within the environment 100 via the network 104. The network 104 can be a global area network (GAN) such as the Internet, a wide area network (WAN), a local area network (LAN), or any other type of network or combination of networks. The network 104 can provide a wireline, wireless, or a combination of wireline and wireless communication between the entities within the system 100.

Each of the data sources 102 is and/or includes one or more computing devices and/or systems that are configured to provide data (e.g., patient data, patient sequencing data, and/or microbiome sequencing data) to the explainability computing system 106. For example, the data sources 102 are and/or include one or more computing devices, computing platforms, systems, servers, desktops, laptops, tablets, mobile devices (e.g., smartphone device, or other mobile device), or any other type of computing device that generally comprises one or more communication components, one or more processing components, and one or more memory components.

The explainability computing system 106 is a computing system that is configured to improve explainability of patient representations in healthcare and hospital management systems. The explainability computing system 106 is and/or includes, but is not limited to, a desktop, laptop, tablet, mobile device (e.g., smartphone device, or other mobile device), server, computing system and/or other types of computing entities that generally comprises one or more communication components, one or more processing components, and one or more memory components.

The database 108 includes EHR 110. The EHR 110 is a systematized collection of patient and/or population electronically stored health information in a digital format. For example, the EHR 110 are records that can be shared across different health care settings. The explainability computing system 106 can retrieve and/or use the records/other information from the EHR 110. In some embodiments, the database 108 further includes a microbiome database. The microbiome database can include information indicating microbiome data associated with one or more patients.

The database 108 is and/or includes, but is not limited to, a storage entity that stores data such as the EHR 110. In some instances, the database 108 can be a repository (e.g., a data repository). In other instances, the database 108 can include a computing device such as a desktop, laptop, tablet, mobile device (e.g., smartphone device, or other mobile device), server, computing system and/or other types of computing entities that generally comprises one or more communication components, one or more processing components, and one or more memory components.

It will be appreciated that the exemplary system depicted in FIG. 1 is merely an example, and that the principles discussed herein may also be applicable to other situations for example, including other types of devices, systems, and network configurations.

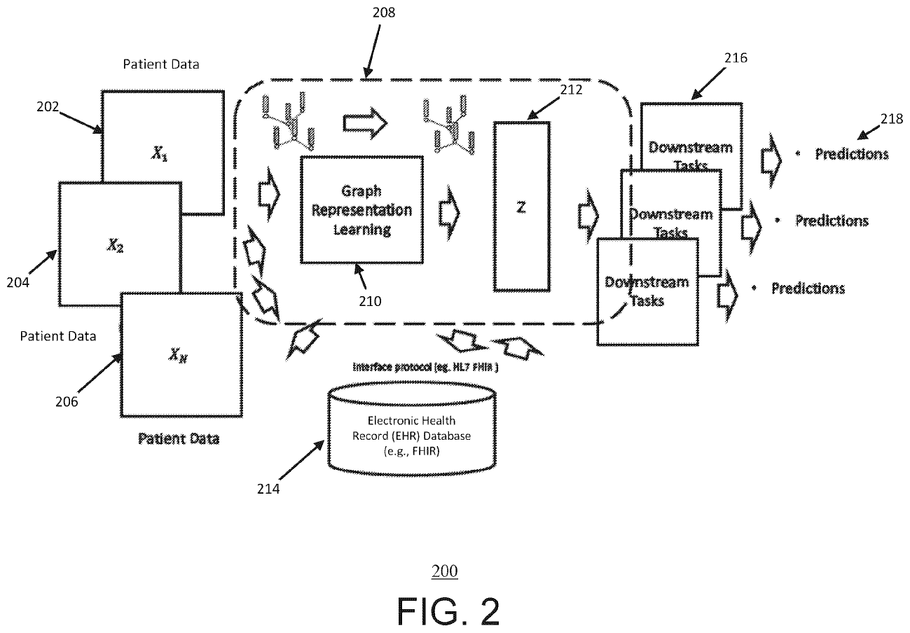

FIG. 2 shows a multi-source, multi-prediction patient representation learning according to an embodiment of the present disclosure. For example, FIG. 2 shows a general setup 200. The general setup 200 addresses the problem of patient representation learning with multiple sources (e.g., patient data 204-206) and multiple downstream prediction tasks (e.g., downstream tasks 216). Referring to FIG. 2, a unique Z 212 is learned by aggregating the input data sources X₁, X₂, . . . X_{N} 202-206.

For example, the data sources 102 can be and/or include the input data sources 202-206 that provide the patient data to the explainability computing system 106. The explainability computing system 106 can perform functionalities such as the functionalities shown in dotted box 208. For instance, the explainability computing system 106 can perform and/or use graph representation learning 210 to learn the unique Z 212. For example, the explainability computing system 106 can aggregate the patient data from the input data sources 202-206 to learn the unique Z 212. The unique Z 212 can be part of, but might not be all of, the explainability, which is described in further detail below. Additionally, and/or alternatively, the explainability computing system 106 can use the database 108, which can be an electronic health record (EHR) database such as a fast healthcare interoperability resources (FHIR) 214. For instance, the explainability computing system 106 can communicate with the FHIR 214 using an interface protocol such as Health Level Seven International (HL7) FHIR protocol. Using the patient data and the EHR database 214, the explainability computing system 106 can perform and/or provide information to other entities (e.g., other computing systems) to perform downstream tasks 216. For instance, the explainability computing system 106 can perform one or more downstream tasks 216 to determine (e.g., make) one or more predictions 218. For example, each downstream task can be a different downstream task, and the explainability computing system 106 can determine separate predictions 218 for each of the downstream tasks 216.

The requirements for the explainable latent representation is described below. For instance, in some examples, embodiments of the present invention can consider the following six requirements for the explainable representation. The first requirement can be a graph of patients, which indicates a representation that is associated with a graph of the patients. The second requirement can be for explainable embedded features (XAI). For instance, from hospital personnel and legislator, the explainable latent representation can be and/or shall be useful for the clinical personnel and help the authority to verify the explainability. The third requirement can include invariant representation using constative loss with graph masking and clustering loss. For instance, embodiments of the present invention can contemplate the use of an invariant representation across multiple predictive tasks and/or use the clustering loss to allow the counterfactual and prototype explainability. The fourth requirement can include a prototype that uses clustering features and/or using virtual nodes. This can be optional. For instance, embodiments of the present invention can contemplate the clustering of the representation to help the prototypical explanation of the features. The fifth requirement can include counterfactuals, which in some embodiments, is optional. For instance, embodiments of the present invention can contemplate dedicated information in the latent feature to help with the counterfactual analysis. The sixth requirement can include missing feature denoise. For instance, embodiments of the present invention can allow the system to reconstruct missing features from the latent representation (e.g., optional with dedicated loss).

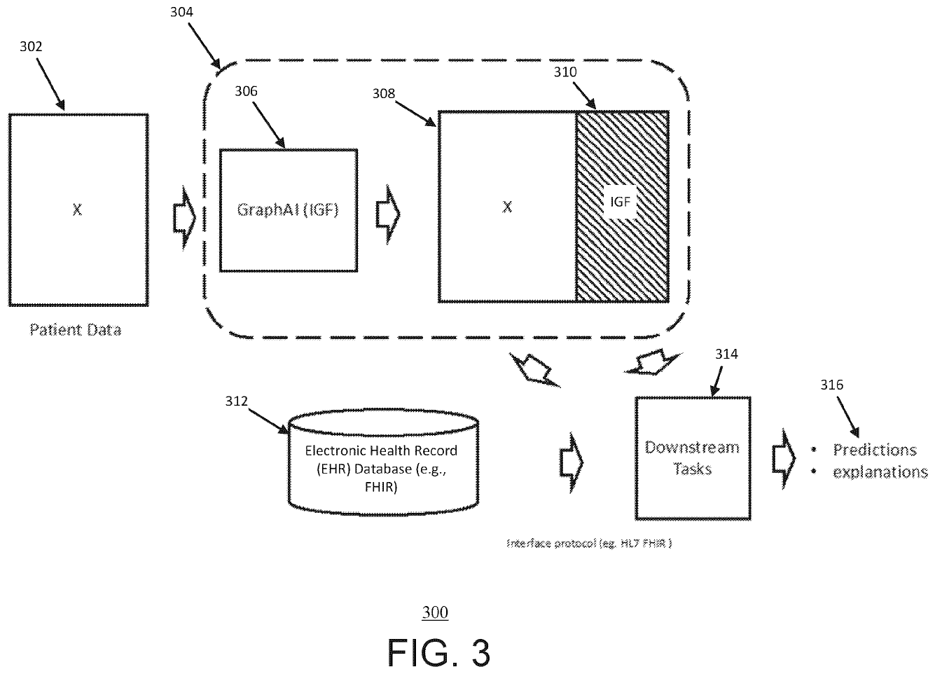

The invariant (and interpretable) graph fingerprint (I2GF) is described below. For example, FIG. 3 shows a high level description of I2GF 300 according to an embodiment of the present disclosure. In order to provide explainable patient representation, embodiments of the present invention build an invariant and explainable representation (e.g., I2GF and/or the invariant graph fingerprint (IGF)) that uses discrete representation features. The discrete nature of the representation allows embodiments of the present invention to consider each dimension separately and evaluate the configuration of the embedding (e.g., latent representation of the patient) by evaluating whether the feature is active or not. Embodiments of the present invention can also ask the system to be invariant to the underlying downstream task, but promote invariant features as well. For instance, the explainability computing system 106 uses the patient data 302 and the graphAI 306 to enhance the input data (e.g., the patient data 302) with IGF/I2GF features 310 (e.g., discrete features). As used herein, IGF and I2GF are used interchangeably. For instance, the enhanced input data includes the patient data 308 (e.g., the same as the patient data 302) and further includes the IGF 310. The enhanced input data can be used for prediction and explanation for the downstream tasks. In some instances, the IGF 310 is the output of the contrastive learning and/or other processing (e.g., the explainability computing system 106 can generate the IGF 310 using contrastive learning and/or other processing techniques). In some variations, the database 312 (e.g., the EHR database such as FHIR) provides the patient data “x” 302. Using the provided patient data “x” 302, the explainability computing system 106 can generate the IGF 310 and provide the IGF 310 back to the database 312. Additionally, and/or alternatively, the database 312 can include the graph G, which can be used by the explainability computing system 106.

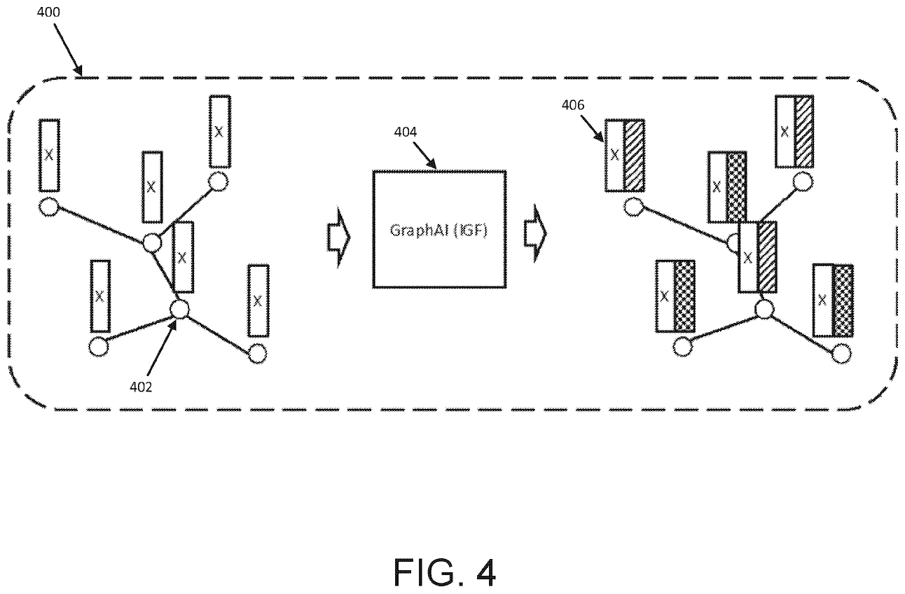

The representation that is learned, called herein as I2GF (e.g., IGF), is then used for the downstream tasks. This is shown in FIG. 4. For example, FIG. 4 shows discrete latent graph feature learning with I2GF according to an embodiment of the present disclosure. For example, the dotted box 400 shows the discrete latent graph feature learning with I2GF. For instance, the graph 402 can indicate the patient data (e.g., the patient data can include and/or be transformed into a graph 402 with nodes that are represented by “x” and edges that connect the nodes to each other). For example, each node of the graph 402 can be associated with patient data (e.g., the patient data 202-206). Based on inputting the patient data into a Graph AI 404, the explainability computing system 106 generates a graph 406.

For instance, the explainability computing system 106 can input the graph 402 into the GraphAI 404 to generate the graph 406. The graph 402 is composed of nodes (e.g., the patients) with their information (e.g., static and dynamic data) and edges. In some instances, the edges include edge attributes and can be computed (e.g., by the explainability computing system 106 and/or another computing system) based on other information. The edges represent if two patients (e.g., the nodes) are related. The output of the GraphAI 404 (e.g., the graph 406) includes IGF features that are added to the nodes of the graph 402. In some embodiments, the graph 406 includes IGF features for the edges of the graph 402 as well.

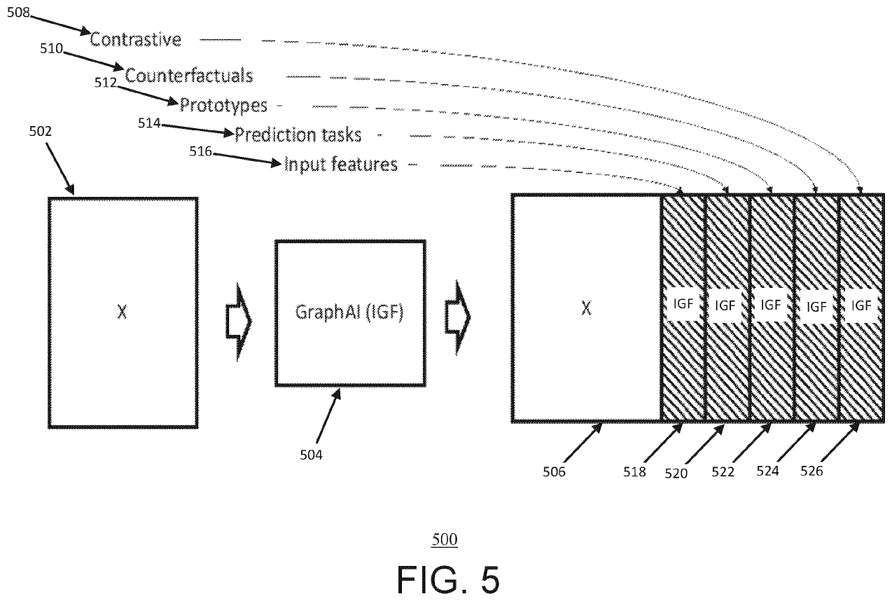

The dedicated reconstruction loss is described below. For instance, to further improve interpretability, embodiments of the present invention consider the case where the discrete latent representation (e.g., the IGFs) is divided, where each feature can be a special loss function and/or be associated with a special loss function during training. For example, embodiments of the present invention thus consider the use of multiple loss to implement the requirements, where the loss can be activated or deactivated to promote accuracy versus explainability according to the system owner. This is described in more detail with respect to FIG. 5. FIG. 5 shows dedicated losses that are used to reconstruct the input features or to promote prototypes, counterfactuals, or the basic contrastive loss according to an embodiment of the present disclosure.

For example, FIG. 5 shows a method 500 that includes the input 502 such as the patient data (e.g., patient data 202-206). The explainability computing system 106 can use the GraphAI (IDG) 504 and the input 502 to generate the outputs 506 and/or 518-526. For example, the output 506 can be associated with the input 502 (e.g., the original patient data). The outputs 518-526 (e.g., the IGFs or the discrete latent representations) can be associated with the features 508-516 (e.g., the contrastive 508, the counterfactuals 510, the prototypes 512, the prediction tasks 514, and the input features 516). Additionally, and/or alternatively, the explainability computing system 106 can use one or more loss functions associated with the IGFs 518-526 to reconstruct the input features or to promote prototypes, counterfactuals, or the basic contrastive loss.

For instance, the explainability computing system 106 can compute (e.g., determine) each additional component of the IGF (e.g., IGFs 518-526) based on a dedicated loss and/or an unsupervised computations. For example, the explainability computing system 106 can compute the IGF 518 for the Input Features 516. The Input Features 516 are features that discrete the input feature (e.g., the features of the input data 502) in a block of categorical features that represent the input feature, and in some embodiments, can still allow the input feature to return back to the original feature for explainability. In some instances, the Input Features 516 are a compressed version of the original features (e.g., features associated with the input 502, which can be the patient data). In some examples, multiple input features can be grouped together. In other examples, the multiple input features might not be grouped together.

The explainability computing system 106 can compute the IGF 520 for the Prediction Task 514. The Prediction Task 514 can include minimal subsets of the input features that allows a prediction for the specific task (e.g., a discrete subset of the input data that is used for a prediction of a specific task). These Prediction Tasks 514 can be computed again (e.g., re-computed), but they can indicate discrete features and/or selected in an end-to-end manner. In some examples, these features (e.g., the Prediction Task 514) are built from the previous features (e.g., the explainability computing system 106 can compute the IGF 520 for the Prediction Task 514 based on previous features), so they can been seen as a selection of the previous features for each of the common prediction tasks. These tasks can be common tasks that are available for each patient, as for example, the prediction of the frequency of visit or the provision of basic medicaments.

The explainability computing system 106 can compute the IGF 522 for the Prototypes 512. The Prototypes 512 can be computed either on the input features (e.g., the input 502) or based on the discrete input features (e.g., the Input Features 516). The IGF 522 for the Prototypes 512 represent a clustering of the input or output features and can be used to compute similarity of the patients (e.g., the patient representations).

The explainability computing system 106 can compute the IGF 524 for the Counterfactuals 510. The counterfactual features 510 are computed based on the vicinity criteria. For instance, this can be the feature that, by changing, can classify the patient to belonging, for example, to another cluster of the Prototype 512 or to a different prediction task's class (e.g., from high risk to medium risk).

The explainability computing system 106 can compute the IGF 526 for the Contrastive 508. The contrastive 508 are the features that are learned based on the contrastive loss. These can be based from, for example, on the first feature or any other features (e.g., the features 518-526).

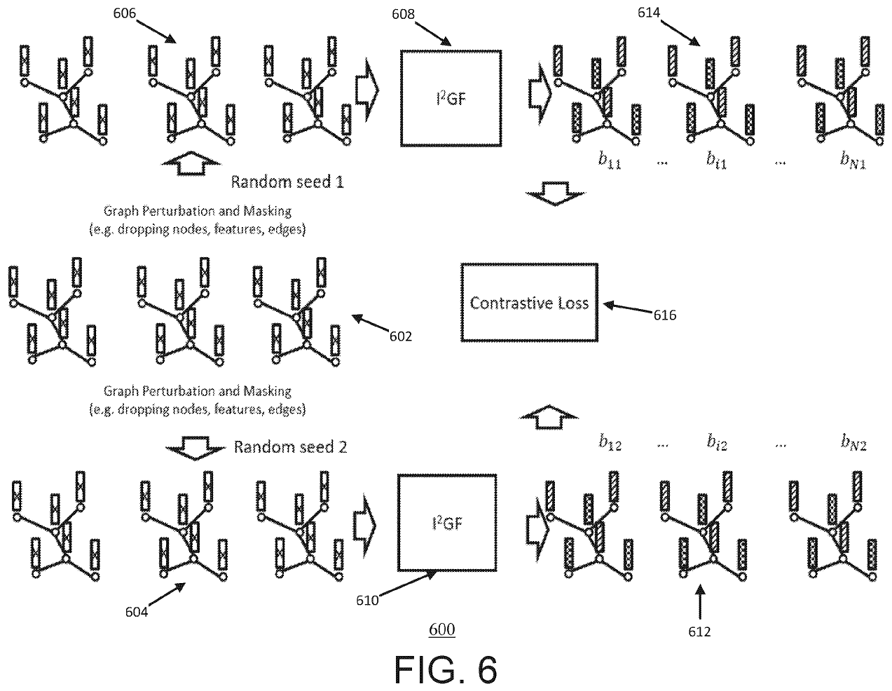

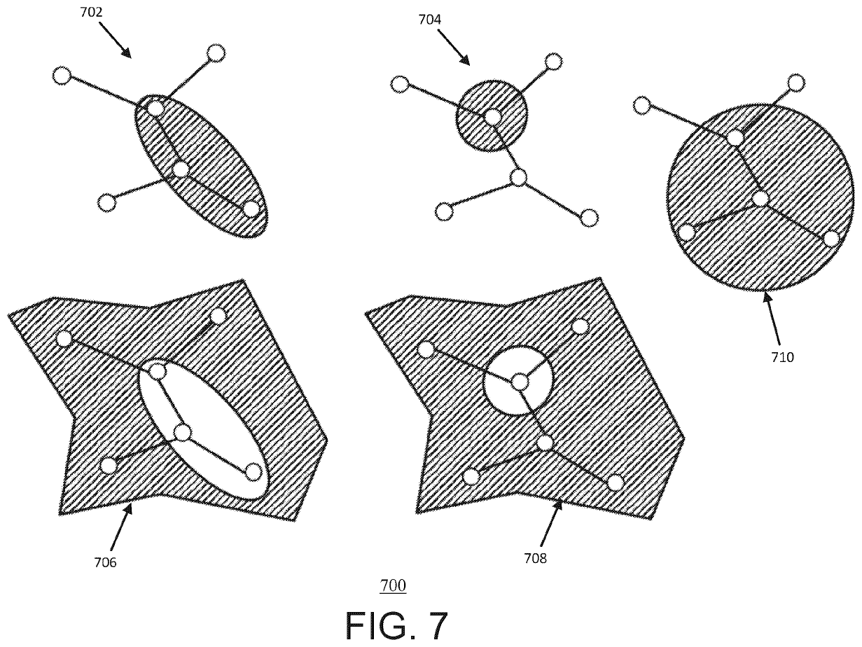

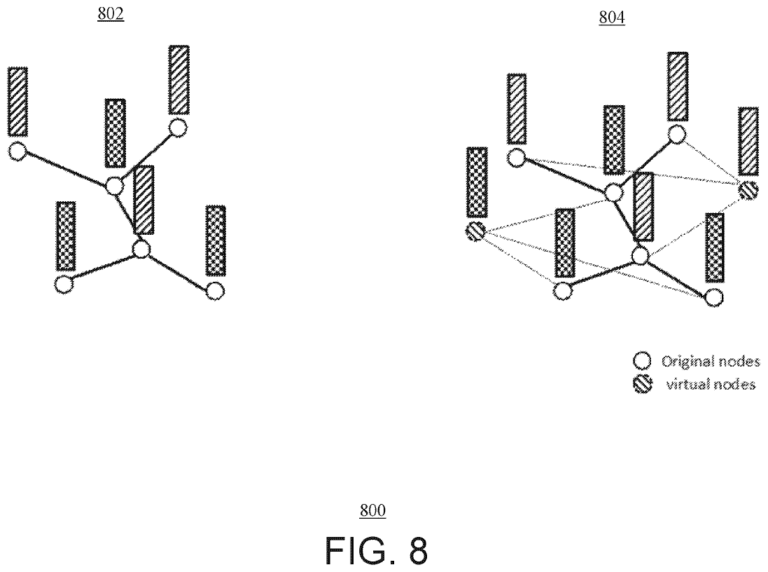

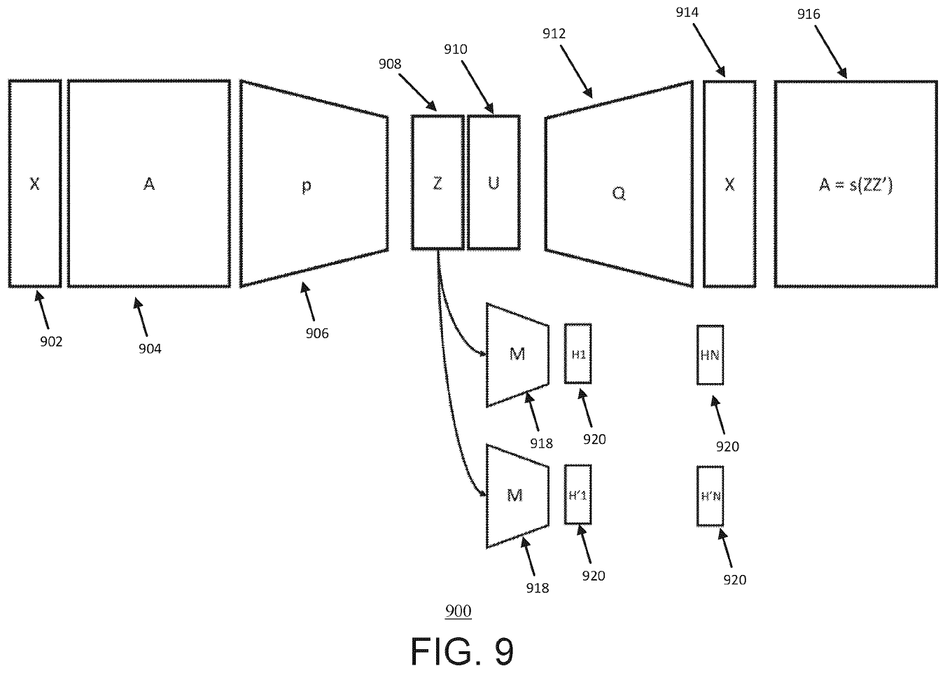

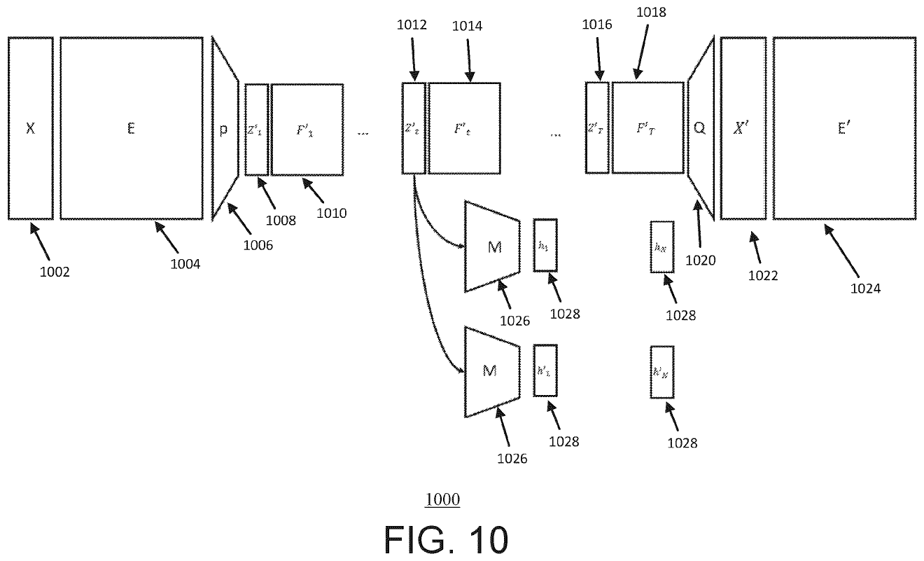

The architecture details are described below. For instance, embodiments of the present invention (e.g., the explainability computing system 106) can consider the following:
1. Projection from continuous to discrete (and reverse when reconstruction is implemented). For instance, this is described by FIG. 5. For example, the explainability computing platform 106 can use the method 500 to project from continuous to discrete (e.g., change the input 502 from continuous to discrete).
2. Generation of perturbed graphs with masking for contrastive learning. For instance, this is described by FIGS. 6 and 7 below.
3. Prototype derived node features: clustering+ (ordered) k-nearest neighbors (knn) algorithm. For instance, this is described by FIG. 8 below.
4. Use prototype for counterfactual, the second closes knn could be a proposal (not possible for representation). For instance, this is described by FIG. 8 below.
5. Split embeddings: 1) if there is any task, add feature to predict only that task, 2) for each input feature add a prediction task associated with a subset of the embedding features. For instance, this is described by FIG. 5 above.
6. (Optional) Discrete Denoising Diffusion Graph Auto-Encoder (see, e.g., Vignac, Clement, et al., “DiGress: Discrete Denoising diffusion for graph generation,” arXiv: 2209.14734 (2022), which is hereby incorporated by reference herein). For instance, this is described by FIGS. 9 and 10 below.
7. (Optional) Discrete Graph Variational Auto-Encoder (e.g., Graph Isomorphism Network (GIN) and/or straight-through (ST) discrete variational autoencoder). For instance, this is described by FIGS. 9 and 10 below.

The graph contrastive loss is described below. For instance, for promoting invariant latent features, embodiments of the present invention (e.g., the explainability computing system 106) can consider the following equations (Eq.) 1-4 for the contrastive losses. For example, the explainability computing system 106 can minimize the Kullback-Leibler (KL) divergence of representation using the below:

$$KL ( {b}_{i 2} \vert {b}_{i 1} ) < KL ( {b}_{i 2} \vert {b}_{j 1} ) , ∀ j \neq i \qquad Eq . 1$$

which can be computed as:

$$min KL ( {b}_{i 2} \vert {b}_{i 1} ) - β {\sum }_{j \neq i} KL ( {b}_{i 2} \vert {b}_{j 1} ) , ∀ j \neq i \qquad Eq . 2$$

where “bi” is the “i” feature, “bj” is the j feature, that is the feature associated with the i and j node (or patient) in the current training batch. The second index (1,2) is which of the two batches are considered. β is a hyper-parameter.

The explainability computing system 106 can perform mutual information maximization using the below:

$$max MI ( {b}_{i 2} ; {b}_{i 1} ) - β {\sum }_{j} MI ( {b}_{j 2} ; {b}_{i 1} ) \qquad Eq . 3$$

where MI represents mutual information, which can be defined as MI(X;Y)=H(X)−H(X|Y).

The explainability computing system 106 can perform maximizing the cosine similarity function σ(bᵢ₂; bᵢ₁) using the below:

$$max \frac{exp σ ( {b}_{i 2} ; {b}_{i 1} ) / τ}{{\sum }_{j \neq i} exp σ ( {b}_{j 2} ; {b}_{i 1} ) / τ} , σ ( {b}_{i} ; {b}_{j} ) = \frac{{b}_{i}^{T} {b}_{j}}{\vert {b}_{i} \vert \vert {b}_{j} \vert} \qquad Eq . 4$$

where σ is a non linear function, which can be a cosine similarity that is defined as σ(x;y)=<x,y>/∥x∥/∥y∥. τ is a temperature hyper-parameter.

The graph perturbation and masking is described below with reference to FIGS. 6 and 7. FIG. 6 shows invariant representation learning with constative loss and graph perturbation and masking 600 according to an embodiment of the present disclosure. For example, for contrastive loss, the explainability computing system 106 uses two streams of input data (e.g., from the data sources 102). For instance, the explainability computing system 106 performs graph perturbation and masking on the graph 602 to generate graphs 604 and 606. The graph perturbation and masking can include, but is not limited to, dropping nodes, features, and/or edges. Additionally, and/or alternatively, random seeds (e.g., random seeds 1 and 2) can be used. The explainability computing system 106 then generates I2GFs 608 and 610 based on the graphs 604 and 606. Then, the explainability computing system 106 generates graphs 612 and 614. The explainability computing system 106 uses graphs 614 and 616 to determine the contrastive loss 616. For example, the explainability computing system 106 can drop the nodes and/or edges to generate the graphs 604 and 606. Then, using the I2GFs 608 and 610 (e.g., Boolean satisfiability problem (SAT) message passing graph neural networks), the explainability computing system 106 determines graphs 612 and 614. Using the Eqs. 1-4 above and the graphs 612 and 614, the explainability computing system 106 determines the contrastive loss. For instance, the explainability computing system 106 can apply Eq. 1 above on graph 614. The explainability computing system 106 can apply Eqs. 3 and 4 on graph 612. Based on applying these Eqs. 1-4, the explainability computing system 106 determines the contrastive loss 616.

For instance, as explained above, the explainability system 106 can determine the contrastive loss based on minimizing the KL divergence of the representation KL(bᵢ₂|bᵢ₁)<KL(bᵢ₂|bⱼ₁), ∀ⱼ≠i, which can be computed as:

$$min KL ( {b}_{i 2} \vert {b}_{i 1} ) - β {\sum }_{j \neq i} KL ( {b}_{i 2} \vert {b}_{j 1} ) , ∀ j \neq i$$

Further, the explainability system 106 can perform mutual information maximization:

$$max MI ( {b}_{i 2} ; {b}_{i 1} ) - β {\sum }_{j} MI ( {b}_{j 2} ; {b}_{i 1} )$$

Then, the explainability system 106 can perform maximizing cosine similarity function σ(bᵢ₂; bᵢ₁)

$$max \frac{exp σ ( {b}_{i 2} ; {b}_{i 1} ) / τ}{{\sum }_{j \neq i} exp σ ( {b}_{j 2} ; {b}_{i 1} ) / τ} , σ ( {b}_{i} ; {b}_{j} ) = \frac{{b}_{i}^{T} {b}_{j}}{\vert {b}_{i} \vert \vert {b}_{j} \vert}$$

For instance, the explainability computing system 106 can generate two sets of graphs 604 and 606 from the original graph 602 based on policies (e.g., any combination of policies). The two graphs 604 and 606 can be represented by: 1) G1, . . . ,GN; and 2) G′1 . . . ,G′N, where G′i=Policy (Gi) is generated according to the policy, Policy ( )

FIG. 7 shows graph generation policies according to an embodiment of the present disclosure. For instance, FIG. 7 shows an environment 700 with multiple graph generation policies 702-710. Further, embodiments of the present invention can consider the following policies:
1. Node, edge and feature masking (removal). For instance, this can refer to randomly removing nodes, edges, and/or nodes/edge features.
2. Around/outside a node i, given a radius b (where distance is measured in # of hop). For instance, graph generation policies 704 (around) and 708 (outside) can provide the node.
3. Between/outside node i and j, given a radius b (where distance is measured in # of hop). For instance, graph generation policies 702 (between) and 706 (outside) can provide the two nodes and connected nodes.
4. Ego-network: sampling a node and take the first b-hops neighbors. For instance, the b-hops neighbors can be represented by graph generation policy 710. In 710, b can equal 1, which can indicate a 1-hop neighbor.

Prototype elements with virtual nodes are described below with reference to FIG. 8. FIG. 8 shows visualization 800 of virtual nodes according to an embodiment of the present disclosure. For instance, embodiments of the present invention (e.g., the explainability computing system 106) can use virtual nodes to implement the prototypes explanations. For example, the explainability computing system 106 can transform the graph 802 into the graph 804 by using the virtual nodes (shown by the shaded nodes in graph 804). The use of the virtual nodes to implement prototype explanations include:
1. Generation of virtual nodes using clustering and adding a clustering loss.
2. Add features based on knn (k-Nearest neighbor algorithm) to virtual nodes (edges)
3. Either one-hot encoded (1 to the closer virtual nodes, 0 for the others) or ordered knn features (id of the closer virtual nodes)

For example, the above corresponds to feature 512 from FIG. 5 (e.g., the Prototypes 512). First, the explainability computing system 106 computes the clusters of the features of the nodes (or the edges) and their cluster head (e.g., the average of the features inside the cluster). As shown, a cluster is a subset of node features that are similar according to a distance. Then, the explainability computing system 106 can add the cluster heads as new nodes (e.g., called the virtual nodes, since there is no real patient that has these features). Following, for each node, the explainability computing system 106 adds a new feature vector that is the distance, which may be thresholded) to the cluster heads or can be the mean of the k-nearest neighbors (k-nn) of this node and the standard deviation. Next, the explainability computing system 106 computes the k-nn and has a binary variable (e.g., one-hot encoding) that states whether the i-th cluster is among the k-neighbor of this node.

The biomarkers are described below. For instance, embodiments of the present invention (e.g., the explainability computing system 106) can be used to detect the biomarkers used in the prediction. For each patient, embodiments of the present invention can predict, for example, the length of stay or the risk of admission to the Intensive Care Unit (ICU) and at the same time, embodiments of the present invention can provide the biomarkers that lead to this prediction, for example, high pressure, low body temperature and high respiratory rate.

By using embodiments of the present invention, this solves the problem of not being able to interpret features of patient representation by creating the biomarkers and detecting the biomarkers (e.g., high pressure, low hearth rate, low body temperature, specific active gene) associated with a specific prediction, or in general, the most important biomarkers for a specific disease.

In some examples, certain technical embodiments can be used by the embodiments of the present invention. For instance, embodiments of the present invention can use a discrete graph variational auto encoder, which is shown in FIG. 9. FIG. 9 shows an alternative architecture for a discrete graph variational auto encoder according to an embodiment of the present disclosure. For instance, the discrete graph variational auto encoder 900 includes a plurality of layers. As shown, X 902: are the input features, Z 908 is the matrix derived from the adjacent matrix of the graph, p 906 is encoding matrix, z part of the embedding, while U 910 is the rest of the embedding. (Z,U) are used to reconstruct the input X via the decoder network Q. A=s (ZZ′) 916 is the reconstructed, normalized adjacent matrix A 904: M 918 is an encoding matrix of the partial feature Z 908, and the output (e.g., H1-HN and H′1-H′N) 920 is used for contrastive loss. This is done for each graph separately and the features are added in FIG. 5, in addition to or as an alternative to the other contrastive features in 508 (e.g., the contrastive feature 508).

Additionally, and/or alternatively, embodiments of the present invention can use a discrete denoising diffusion graph auto-encoder (e.g., a discrete diffusion model), which is shown in FIG. 10. FIG. 10 shows a discrete diffusion model according to an embodiment of the present disclosure. For example, the discrete diffusion model 1000 includes a plurality of layers.

For instance, similar to the auto encoder version, the diffusion, working in the embedded space using a diffusion model generates the feature X 1002 and the edges E 1004 from noise. The neural network p 1006 and Q 1020 represent the encoder and decoder that are trained separately as auto encoders. The diffuse state X′, E′ 1022 and 1024 are used for the contrastive learning, similar to the auto encoder. The X, E in the middle (e.g., the blocks 1008-1018) are the latent variable associate to X, E and are generated stated from noise. M 1026 is a neural network that encodes the features used in the contrastive loss. The output of the feature 1028 is then use as features in FIG. 5 in the contrastive features category.

Additionally, and/or alternatively, embodiments of the present invention can use Shapley importance explanations. For instance, embodiments of the present invention (e.g., the explainability computing system 106) can provide explanations to the user of the system based on the importance computation of the downstream task according to the Shapley prediction to the IGF features. For instance, the Shapley importance is computed based on the contribution of the single variables, so in this case, the explainability computing system 106 can compute the shapely values based on the contribution of the various terms in FIG. 5, both at the category view (which feature class) and inside each category, for each single discrete feature.

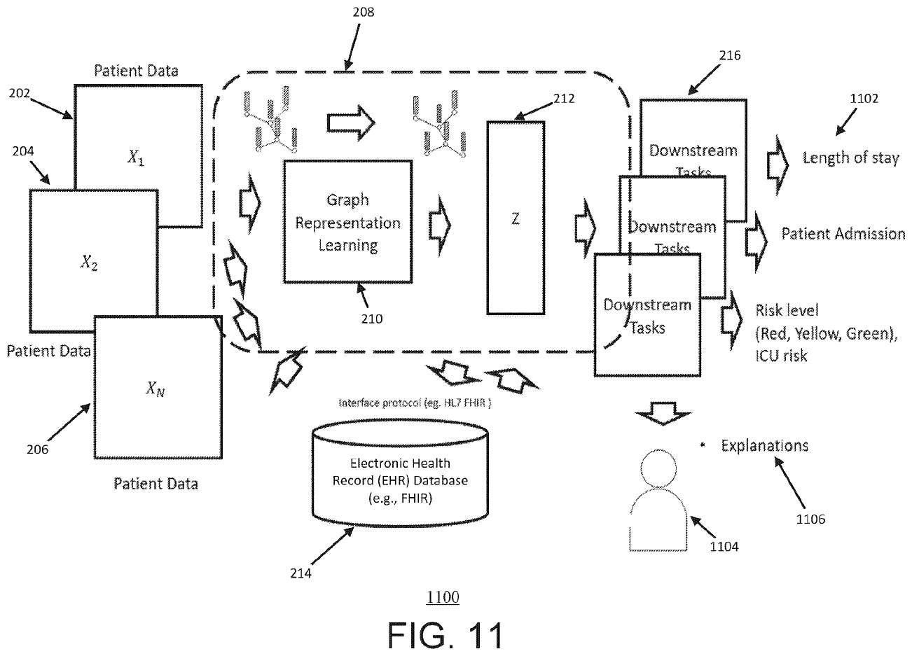

In one or more embodiments, the present invention can be applied to electronic health records (EHR) for length of staying prediction and risk prediction. This will be described with reference to FIG. 11. FIG. 11 shows an IGF that is used for prediction of length of stay and to provide additional information (e.g., justification or explanations) to a hospital personnel according to an embodiment of the present disclosure. For instance, the clinical environment 1100 shows entities similar to the general setup 200 from FIG. 2. For example, the patient data 202-206, the dotted line 208, the graph representation learning 210, the Z 212, the downstream tasks 216, and the EHR database 214 are shown. Further, the clinical environment 1100 shows the predictions 1102 such as length of stay, patient admission, risk level (red, yellow, green), ICU risk. Further, the clinical environment 1100 includes a user 1104 (e.g., a doctor) and explanations 1106.

For example, in the context of a clinical environment (e.g., clinical environment 1100), embodiments of the present invention (e.g., the explainability computing system 106) can be used to provide explainable predictions on the length of staying of a patient in the hospital ward. For instance, a new patient enters the hospital, and his records are added to the pre-existent EHR (e.g., patient data such as patient data 206 can be added to the EHR database 214). The patient (e.g., the patient data 206) is added as a node to the graph of patients, by performing a distance calculation based on the values of the available features. For instance, the explainability computing system 106 can perform a distance calculation based on the values of the available features to add the patient as a node to the graph of patients.

Then, an IGF is run on the complete graph, and a predictive downstream model allows a determination (e.g., prediction) as to how long the patient will remain in the ward. For example, the explainability computing system 106 can input the new graph into the graph representation learning 210 to generate Z 212 (e.g., a predictive downstream model). Then, the explainability computing system 106 can using the predictive downstream model for one or more downstream tasks 216 such as predicting how long the patient will remain in the ward (e.g., length of stay of the patient). The explainability computing system 106 can output (e.g., provide for display) the predictions onto a display device (e.g., a display device associated with the explainability computing system 106). The doctor (e.g., the user 1104) can read the value on a screen together with the variables that justify the choice of that duration. The same graph embedding can be used for predicting the risk of been admitted to the ICU (Intensive Care Unit) or to be dismissed by the ward. For instance, the explainability computing system 106 can use the same graph embedding for other downstream tasks 216/predictions 1102 such as risk level for being admitted to ICU (e.g., red, yellow, green risk level for ICU) and/or patient admission/dismissal.

Additionally, and/or alternatively, embodiments of the present invention (e.g., the explainability computing system 106) generates the biomarkers associated with the patients and then detects the important biomarkers (e.g., high pressure, high respiratory rate, low body temperature, specific gene activation and expression) that caused the specific risk prediction (as the need to ICU admission).

With the prediction of length of stay, embodiments of the present invention can provide the causes or most relevant features (e.g., the biological values that causes to stay longer: a longer length of stay can be associated with higher probability of infections or the occurrence of complications).

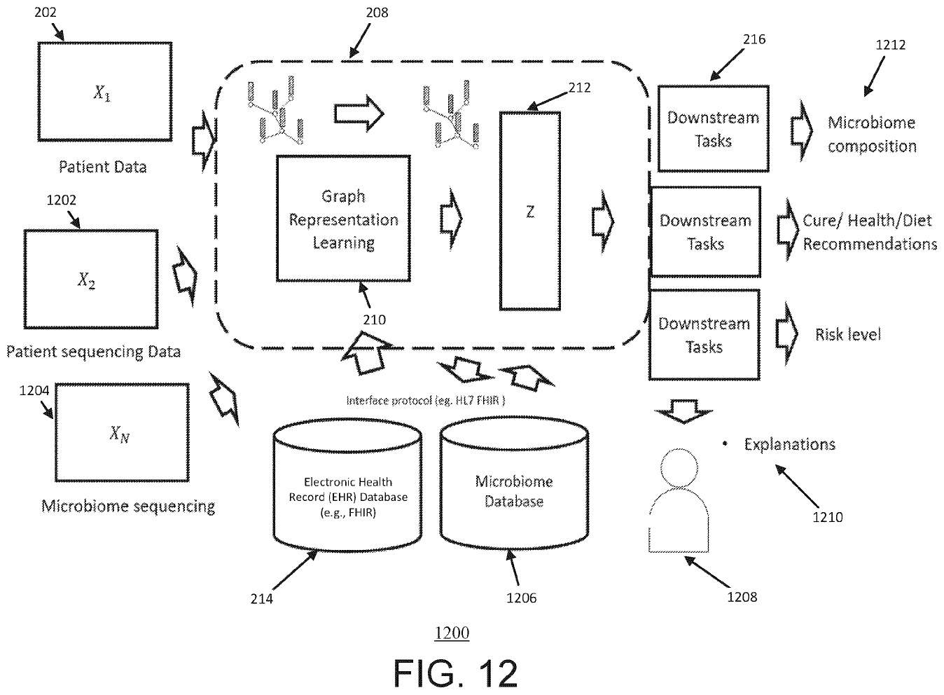

In one or more embodiments, the present invention can be applied to microbiomes. This will be described with reference to FIG. 12. FIG. 12 shows an IGF that is used for predicting a disease of the patient in a multiclass classification task using a microbiome database according to an embodiment of the present disclosure. For instance, the microbiome environment 1200 shows entities similar to the general setup 200 from FIG. 2. For example, the patient data 202, the dotted line 208, the graph representation learning 210, the Z 212, the downstream tasks 216, and the EHR database 214 are shown. Further, the microbiome environment 1200 shows the predictions 1212 such as microbiome composition, cure/health/diet recommendations, and risk level. Further, the microbiome environment 1200 includes patient sequencing data 1202, microbiome sequencing 1204, microbiome database 1206, a user 1208 (e.g., a doctor), and explanations 1210.

For instance, in the context of microbiome (e.g., the microbiome environment 1200), embodiments of the present invention can determine which bacterial species contributes to the development of disease. For instance, a laboratory (e.g., the explainability computing system 106) that performs analysis on microbiome data of the patient can receive a genetic sequencing of the microbiota of a patient (e.g., patient sequencing data 1202 and/or microbiome sequencing 1204). The laboratory (e.g., the explainability computing system 106) already owns a database (e.g., microbiome database 1206) of microbiota from different patients, together with the associated disease (or healthy status). A graph is generated from this data, where each node contains the gene expression of the different bacteria. For instance, based on the patient data 202, the patient sequencing data 1202, and the microbiome sequencing 1204, the explainability computing system 106 can generate a graph that includes nodes comprising the gene expression of the different bacteria. IGF can be used to predict the disease of the patient in a multiclass classification task, and provide the most important feature that contribute to the disease. For example, the explainability computing system 106 can use the graph representation learning 210 to generate Z 212. For instance, using the IGF, the explainability computing system 106 can predict the disease of the patient in a multiclass classification task. Embodiments of the present invention can be used to identify which bacteria species are causing or associated to a specific disease of a patient. For example, the explainability computing system 106 can determine predictions 1212 such as the microbiome composition, cure/health/diet recommendations, and risk level. The explainability computing system 106 can provide for display (e.g., on a display device) the explanations 1210 to a user 1208 (e.g., doctor).

In an embodiment, the present invention provides a method for improving explainability of patient representations in Healthcare and Hospital management Systems, comprising the steps of:
1. Collect data from patients from different subsystems in the hospital, for example to create an EHR system
2. Generate the patient representation according to the inventive step 1 below, with discrete features
3. Use the generated features for downstream tasks; for example, embodiments of the present invention can generate the biomarkers (features) that are then used in the prediction
4. Train a model on the provided biomarkers
5. Predict the risk for a specific patient, using the trained model and detect the biomarkers that led to the specific prediction. Provide explanations to the hospital personnel, to patients or users of the system, where the explanations are connected to the IGF features (e.g., importance of the features according to the Shapley explanations)

Embodiments of the present invention provide for the following improvements over existing technology:
1) Building invariant feature representation for patient of a hospital that are used as explanation for the downstream tasks: generating the biomarkers that are the used during the prediction to detect which biomarkers are the cause of the specific patient prediction:
   a. where the feature is composed of discrete (categorical) variables to have more interpretable explanations
   b. that is connected to input features
   c. that represent prototype patients
   d. that represent the performance on pre-defined downstream tasks
   e. that support the counterfactual reasoning, e.g., closed features in the input space that bring to a different classification.

In some examples, embodiments of the present invention allows the capability to have multiple downstream tasks performed on the graph representations, without having to execute (e.g., run) the representation learning model that might not have access while still providing explanations of the prediction.

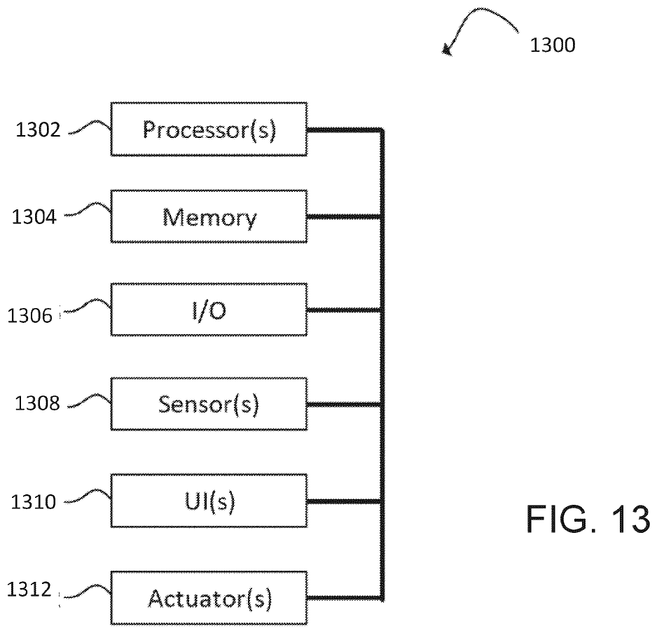

FIG. 13 is a block diagram of an exemplary processing system, which can be configured to perform any and all operations disclosed herein. Referring to FIG. 13, a processing system 1300 can include one or more processors 1302, memory 1304, one or more input/output devices 1306, one or more sensors 1308, one or more user interfaces 1310, and one or more actuators 1312. Processing system 1300 can be representative of each computing system disclosed herein.

Processors 1302 can include one or more distinct processors, each having one or more cores. Each of the distinct processors can have the same or different structure. Processors 1302 can include one or more central processing units (CPUs), one or more graphics processing units (GPUs), circuitry (e.g., application specific integrated circuits (ASICs)), digital signal processors (DSPs), and the like. Processors 1302 can be mounted to a common substrate or to multiple different substrates.

Processors 1302 are configured to perform a certain function, method, or operation (e.g., are configured to provide for performance of a function, method, or operation) at least when one of the one or more of the distinct processors is capable of performing operations embodying the function, method, or operation. Processors 1302 can perform operations embodying the function, method, or operation by, for example, executing code (e.g., interpreting scripts) stored on memory 1304 and/or trafficking data through one or more ASICs. Processors 1302, and thus processing system 1300, can be configured to perform, automatically, any and all functions, methods, and operations disclosed herein. Therefore, processing system 1300 can be configured to implement any of (e.g., all of) the protocols, devices, mechanisms, systems, and methods described herein.

For example, when the present disclosure states that a method or device performs task “X” (or that task “X” is performed), such a statement should be understood to disclose that processing system 1300 can be configured to perform task “X”. Processing system 1300 is configured to perform a function, method, or operation at least when processors 1302 are configured to do the same.

Memory 1304 can include volatile memory, non-volatile memory, and any other medium capable of storing data. Each of the volatile memory, non-volatile memory, and any other type of memory can include multiple different memory devices, located at multiple distinct locations and each having a different structure. Memory 1304 can include remotely hosted (e.g., cloud) storage.

Examples of memory 1304 include a non-transitory computer-readable media such as RAM, ROM, flash memory, EEPROM, any kind of optical storage disk such as a DVD, a Blu-Ray R disc, magnetic storage, holographic storage, a HDD, a SSD, any medium that can be used to store program code in the form of instructions or data structures, and the like. Any and all of the methods, functions, and operations described herein can be fully embodied in the form of tangible and/or non-transitory machine-readable code (e.g., interpretable scripts) saved in memory 1304.

Input-output devices 1306 can include any component for trafficking data such as ports, antennas (i.e., transceivers), printed conductive paths, and the like. Input-output devices 1306 can enable wired communication via USBR, Display Port®, HDMI®, Ethernet, and the like. Input-output devices 1306 can enable electronic, optical, magnetic, and holographic, communication with suitable memory 1304. Input-output devices 1306 can enable wireless communication via WiFiR, Bluetooth®, cellular (e.g., LTE®, CDMA®, GSM®, WiMax®), NFC®, GPS, and the like. Input-output devices 506 can include wired and/or wireless communication pathways.

Sensors 1308 can capture physical measurements of environment and report the same to processors 1302. User interface 1310 can include displays, physical buttons, speakers, microphones, keyboards, and the like. Actuators 1312 can enable processors 1302 to control mechanical forces.

Processing system 1300 can be distributed. For example, some components of processing system 1300 can reside in a remote hosted network service (e.g., a cloud computing environment) while other components of processing system 1300 can reside in a local computing system. Processing system 1300 can have a modular design where certain modules include a plurality of the features/functions shown in FIG. 13. For example, I/O modules can include volatile memory and one or more processors. As another example, individual processor modules can include read-only-memory and/or local caches.

In some instances, the sensors 1308 can be used to populate the EHR database 214 described above. For instance, the sensors 1308 can be used to measure the blood pressure and/or the heath rate, and the measurements can be used to populate the EHR database 214. The UIs 1310 can be used as the component for visualizing the explanations described above. The following is also incorporated by reference herein in its entirety:
A. Duval and F. D. Malliaros, “GraphSVX: Shapley Value Explanations for Graph Neural Networks.” arXiv, Jul. 13, 2021. doi: 10.48550/arXiv.2104.10482.
Wang, J. Wiens, and S. Lundberg, “Shapley Flow: A Graph-based Approach to Interpreting Model Predictions,” in Proceedings of The 24th International Conference on Artificial Intelligence and Statistics, March 2021, pp. 721-729. Accessed: Mar. 6, 2023. [Online]. Available: https://proceedings.mlr.press/v130/wang21b.html.
U.S. Patent Application Publication No. US20170046602A1, titled, “Learning temporal patterns from electronic health records”, and filed on Oct. 23, 2015.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A computer-implemented method for improving explainability of patient representations, comprising:
generating one or more patient representations of a patient based on building one or more invariant feature representations of the patient, wherein the one or more patient representations indicate one or more discrete features:
determining predictions for one or more downstream tasks based on using the one or more discrete features; and
providing explanations associated with the one or more discrete features, wherein the explanations are associated with the predictions for the one or more downstream tasks.

2. The method of claim 1, further comprising:
collecting data from a plurality of patients from different subsystems within a hospital environment; and
creating an electronic health record (EHR) database based on the collected data, wherein generating the one or more patient representations is based on using the EHR database.

3. The method of claim 1, wherein generating the one or more patient representations of the patient comprises generating biomarkers for the patient, wherein determining the predictions for the one or more downstream tasks is based on the generated biomarkers.

4. The method of claim 3, further comprising:
training a model based on the biomarkers for the patient, wherein determining the predictions is based on the trained model.

5. The method of claim 4, further comprising:
predicting one or more risks for the patient based on using the trained model; and
detecting, based on the one or more risks, specific biomarkers from the generated biomarkers that cause each of the predictions, wherein the explanations indicate the predictions and the specific biomarkers that caused the predictions.

6. The method of claim 1, wherein providing, for display, the explanations comprises providing the explanations for display on a hospital display device associated with hospital personnel, one or more patients, or other users.

7. The method of claim 1, wherein the one or more discrete features comprise invariant graph fingerprint (IGF) features, wherein the explanations are associated with the IGF features, and wherein the explanations indicate importance of the IGF features according to Shapley importance explanations.

8. The method of claim 1, wherein generating the one or more patient representations of the patient comprises determining a first invariant graph fingerprint (IGF) feature for input features based on using a graph artificial intelligence (Graph AI) and input data, wherein the first IGF feature is a discrete version of the input data.

9. The method of claim 1, wherein generating the one or more patient representations of the patient comprises determining, based on using the Graph AI and the input data, a second IGF feature for prediction tasks and a third IGF feature for prototypes, wherein the second IGF feature is a discrete subset of the input data that is used for a prediction of a specific task, and wherein the third IGF feature indicates a clustering of the input data associated with similarities between the one or more patient representations.

10. The method of claim 9, wherein determining the third IGF feature for prototypes is based on using one or more generated virtual nodes and adding features that are determined using a k-Nearest neighbor algorithm.

11. The method of claim 1, wherein generating the one or more patient representations of the patient comprises determining a fourth IGF feature for counterfactuals and determining a fifth IGF feature for a contrastive associated with a contrastive loss.

12. The method of claim 11, wherein the contrastive loss is associated with minimizing the Kullback-Leibler (KL) divergence, performing mutual information maximization, and/or maximizing the cosine similarity function.

13. The method of claim 1, wherein generating the one or more patient representations of the patient comprises determining one or more IGF features based on using a dedicated loss or one or more unsupervised computations.

14. A computer system for improving explainability of patient representations, the system comprising one or more hardware processors, which, alone or in combination, are configured to provide for execution of the following steps:
generating one or more patient representations of a patient based on building one or more invariant feature representations of the patient, wherein the one or more patient representations indicate one or more discrete features:
determining predictions for one or more downstream tasks based on using the one or more discrete features; and
providing explanations associated with the one or more discrete features, wherein the explanations are associated with the predictions for the one or more downstream tasks.

15. A tangible, non-transitory computer-readable medium having instructions thereon which, upon being executed by one or more processors, alone or in combination, provide for execution of a method for improving explainability of patient representations comprising the following steps:
generating one or more patient representations of a patient based on building one or more invariant feature representations of the patient, wherein the one or more patient representations indicate one or more discrete features:
determining predictions for one or more downstream tasks based on using the one or more discrete features; and
providing explanations associated with the one or more discrete features, wherein the explanations are associated with the predictions for the one or more downstream tasks.
